SEQUENCE LISTING (23 sequences)

SEQ ID NO: 1 - protein, 279 aa, Homo sapiens
WTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCK

SEQ ID NO: 2 - protein, 15 aa, Artificial Sequence
  CDR light chain amino acid sequence
KASQSVDYDGHSYMN

SEQ ID NO: 3 - protein, 7 aa, Artificial Sequence
  CDR light chain amino acid sequence
AASNLES

SEQ ID NO: 4 - protein, 9 aa, Artificial Sequence
  CDR light chain amino acid sequence
QQSDENPLT

SEQ ID NO: 5 - protein, 112 aa, Artificial Sequence
  Mature light chain variable region amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 6 - protein, 5 aa, Artificial Sequence
  CDR heavy chain amino acid sequence
SYDIN

SEQ ID NO: 7 - protein, 17 aa, Artificial Sequence
  CDR heavy chain amino acid sequence
WIYPGDGSIKYNEKFKG

SEQ ID NO: 8 - protein, 13 aa, Artificial Sequence
  CDR heavy chain amino acid sequence
RGEYGNYEGAMDY

SEQ ID NO: 9 - protein, 122 aa, Artificial Sequence
  Mature heavy chain variable region amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 10 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 218 aa, Artificial Sequence
  Light chain mature amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 448 aa, Artificial Sequence
  Heavy chain mature amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 238 aa, Artificial Sequence
  Light chain complete amino acid sequence
MESQTQVFVYMLLWLSGVDGDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 467 aa, Artificial Sequence
  Heavy chain complete amino acid sequence
MKCSWVIFFLMAVVTGVNSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16-17 - skipped (2 entries)

SEQ ID NO: 18 - protein, 132 aa, Artificial Sequence
  Complete light variable region amino acid sequence
MESQTQVFVYMLLWLSGVDGDIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 19 - protein, 141 aa, Artificial Sequence
  Complete heavy variable region amino acid sequence
MKCSWVIFFLMAVVTGVNSQVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 20-22 - skipped (3 entries)

SEQ ID NO: 23 - protein, 326 aa, Artificial Sequence
  IgG2 constant region amino acid sequence with a one amino acid residue mutation to reduce complement activation
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2022/0324983 A1
(43) Pub. Date: Oct. 13, 2022

(54) USE OF THE ANTI-P-SELECTIN ANTIBODY CRIZANLIZUMAB FOR TREATING SICKLE CELL NEPHROPATHY AND CHRONIC KIDNEY DISEASE ASSOCIATED WITH SICKLE CELL DISEASE

(71) Applicant: NOVARTIS AG, Basel (CH)

(72) Inventors: Kenneth ATAGA, Memphis, TN (US); Laurie DEBONNETT, East Hanover, NJ (US); Vimal DEREBAIL, Chapel Hill, NC (US); Guangyang HAN, East Hanover, NJ (US); Adlette INATI, Tripoli (LB); Julie KANTER, Birmingham, AL (US); Jeffrey LEBENSBURGER, Birmingham, AL (US); Santosh SARAF, Chicago, IL (US); Claire SHARPE, London (GB); Pablo BARTOLUCCI, Créteil (FR); Miona STANKOVIC, Basel (CH)

(21) Appl. No.: 17/633,893

(22) PCT Filed: Aug. 6, 2020
(86) PCT No.: PCT/IB2020/057438

§ 371 Date: Feb. 8, 2022

Related U.S. Application Data
(60) Provisional application No. 62/884,313, filed on Aug. 8, 2019.

(51) Int. Cl. C07K 16/28; A61P 13/12; A61K 45/06; A61K 39/395

(52) U.S. Cl. CPC C07K 16/2854; A61P 13/12; A61K 45/06; A61K 39/3955; A61K 2039/505

(57) ABSTRACT

The invention relates to a method of treating chronic kidney disease due to sickle cell nephropathy in a patient in need of such treatment, comprising administering a pharmaceutically effective amount of an anti-P-selectin antibody or a binding fragment thereof to said patient and related invention embodiments (uses, methods, pharmaceutical preparations and use in the preparation of pharmaceutical preparations).

### FIELD OF INVENTION

The invention relates to the treatment of chronic kidney disease (CKD), especially in patients with Sickle cell Disease (SCD), with a drug, especially an antibody, capable of binding to P-selectin. Methods of treatment, uses, pharmaceutical preparations, their manufacture and the drug for use in treatment of CKD, respectively, are examples of specific embodiments of the invention.

### BACKGROUND

Sickle cell disease (SCD) is a rare autosomal recessive blood disorder caused by a single missense mutation (Glu6Val) in the β-globin gene. The most severe forms of SCD are homozygous hemoglobin (Hb) S (sickle cell anemia) (HbSS) and the heterozygous form Hb S (HbS) with β-thalassemia (HbSβ0-thal) (Ware et al 2017).

Sickle cell nephropathy (SCN) refers to the spectrum of renal complications in sickel cell disease (SCD) and is considered one of the most severe complications of SCD. The renal consequences of SCD manifest in early childhood and progress thereafter. When abnormalities of kidney structure or function are present for more than 3 months, with implications for health, CKD is diagnosed (KDIGO 2013). The prevalence of chronic kidney disease (CKD) in patients with SCD increases with age and progresses to end-stage renal disease in around 12% of patients with SCD (Powars et al 2005; Gosmanova et al 2014). Results of one study demonstrated that acute kidney injury (AKI) occurred in approximately 46% of patients with HbSS and HbSβ0-thal, and AKI and AKI severity are independent risk factors for CKD progression (Saraf et al 2018).

Development of SCN is complex and several pathophysiological mechanisms have been proposed to explain its development. Vaso-occlusion and hemolysis contribute substantially to the manifestations of SCN, which include glomerulopathies (such as hyperfiltration and proteinuria/albuminuria), hematuria, and tubular defects (Schnog et al 2004; Nasr et al 2006; Sharpe and Thein 2014; Hariri et al 2018). Focal segmental glomerulosclerosis (FSGS) and its variants are the major glomerular lesions.

Microalbuminuria (30-300 milligrams [mg]/gram creatinine) is the earliest clinically detectable symptom of glomerular injury in patients with SCD (Bartolucci et al 2016). When assessed either as microalbuminuria or macroalbuminuria (>300 mg/gram creatinine), proteinuria occurs in approximately 20% of patients in the first two decades, and up to 68% of older patients with HbSS (Guasch et al 2006; McPhearson et al 2011; Bartolucci et al 2016). Albuminuria is more common among patients with severe SCD genotypes (HbSS and HbSβ0-thal) than milder genotypes (Guasch et al 2006; Derebail et al 2019). It is a strong predictor of subsequent renal failure (Sasongko et al 2015).

Patients with albuminuria and SCD exhibit increased urinary excretion markers of tubular injury (kidney injury molecule-1 [KIM-1] and N-acetyl-β-D-glycosaminidase [NAG]) (Nath and Hebbel 2015).

Hyperfiltration is another early indicator of SCN. It manifests as an increase in the estimated glomerular filtration rate (eGFR) to >130 milliliter (mL)/min/1.73 m2 in women and >140 mL/min/1.73 m2 in men (Hirschberg 2010). eGFR is estimated from serum creatinine levels. The recommended equation for calculating eGFR is the CKD Epidemiology Collaboration (CKD-EPI) formula, which matches the accuracy of the Modification of Diet in Renal Disease (MDRD) equation at glomerular filtration rates (GFRs)<60 mL/min/1.73 m2 and offers greater accuracy at higher GFRs (Florkowski and Chew-Harris 2011).

There are no prospective randomized data demonstrating a long-term benefit of any treatments for CKD due to SCN and no treatments are approved for this indication. Thus treatment for CKD due to SCN is currently based on data obtained from clinical situations outside of SCD and typically consists of angiotensin-converting enzyme (ACE) inhibitors, angiotensin-receptor blockers (ARBs) and/or hydroxyurea (HU)/hydroxycarbamide (HC). Thus there is strong need to develop medicament effectively preventing or treating SCN, especially CKD within SCN.

The presence of P-selectin expression in the kidneys has been established based on in vitro and in vivo data, and there is evidence that P-selectin is upregulated in the kidney in response to renal ischemia-reperfusion injury in SCD (Zizzi et al 1997; Koo et al 1998; Singbartl et al 2000; Tam 2002). The authors of a pre-clinical study concluded that P-selectin was necessary to produce severe acute renal failure in response to ischemia-reperfusion. Expression of P-selectin in a glomerulonephritis induced mouse model was associated with rapid accumulation of neutrophils in glomeruli and significant proteinuria. P-selectin inhibition in this model was shown to abrogate glomerular neutrophil accumulation and prevented development of proteinuria (Tipping et al 1994). Though this study provides pre-clinical evidence that P-selectin expression is linked to proteinuria, it is unknown if blocking P-selectin in the renal vasculature in vivo will have a beneficial impact on glomerulopathy and will delay the progression of CKD.

In SUSTAIN clinical trial, treatment of SCD patients with crizanlizumab at 5.0 mg/kg showed positive clinical activity as demonstrated by a statistically significant and clinically relevant decrease in the annual VOC rate compared with placebo and it was also found to be well tolerated (Ataga et al 2017).

### DESCRIPTION OF THE INVENTION

It is important to note that the SUSTAIN population was unselected with respect to CKD and approximately 20% of patients had proteinuria at baseline. Patients with any SCD genotype history could be eligible for SUSTAIN. Therefore, the SUSTAIN population was not consistent with a population in which crizanlizumab could be expected to provide a benefit by reducing proteinuria/albuminuria. A population for CKD study ideally would consist of patients with severe SCD genotypes who have albuminuria and a rapid eGFR decline. Patients with hyperfiltration would preferably need to be excluded to avoid confounding the study results. The efficacy in treating CKD by blocking P-seletin, especially by crizanlizumab, is expected to be demonstrated through the inventive trial design, especially through careful selection of SCD patient population and through the identification of the relevant endpoints. The study design can demonstrate a positive effect of crizanlizumab on albumin to creatinine ratio (ACR) reduction and the progression of CKD. This is thought to provide positive evidence of a reno-protective effect of crizanlizumab.

The present invention provides a method of treating or preventing sickel cell nephropathy (SCN) in a patient in need of such treatment, comprising administering a pharmaceutically effective amount of an anti-P-selectin antibody or a binding fragment thereof, especially crizanlizumab, to said patient.

The present invention provides a method of treating or preventing chronic kidney disease (CKD) due to sickel cell nephropathy (SCN) in a patient in need of such treatment, comprising administering a pharmaceutically effective amount of an anti-P-selectin antibody or a binding fragment thereof, especially crizanlizumab, to said patient.

The present invention further provides an anti-P-selectin antibody or a binding fragment thereof, especially crizanlizumab, for use in the treatment or prevention of sickel cell nephropathy (SCN).

The present invention further provides an anti-P-selectin antibody or a binding fragment thereof, especially crizanlizumab, for use in the treatment or prevention of CKD due to sickel cell nephropathy (SCN).

The present invention further provides an anti-P-selectin antibody or a binding fragment thereof, especially crizanlizumab, for use in the treatment or prevention of CKD due to sickel cell nephropathy (SCN).

The present invention further provides an anti-P-selectin antibody or a binding fragment thereof, especially crizanlizumab, for use in the treatment or prevention of CKD due to sickel cell nephropathy (SCN), where said patient has albuminuria (ACR) decrease by at least 20%, by at least 25%, preferably by at least 30%, preferably by at least 35%, preferably by at least 40% compared to the ACR level at baseline (before patient being treated by anti-P-selectin antibody or a binding fragment thereof, especially crizanlizumab). In one embodiment the ACR is measured at 3 m, 6 m, 9 m or 12 m after the treatment of anti-P-selectin antibody or a binding fragment thereof, especially crizanlizumab, preferably according to the dosing schedule of the invention. In one embodiment the ACR is measured at 12 m after the treatment.

The present invention further provides an anti-P-selectin antibody or a binding fragment thereof, especially crizanlizumab, for use in the treatment or prevention of CKD due to sickel cell nephropathy (SCN), where said patient has decrease of PCR by at least 10%, preferably by at least 15%, at least 20%, at least 25%, preferably at least 30% compared to the value of PCR at baseline. In one embodiment the PCR is measured at 3 m, 6 m, 9 m or 12 m after the treatment of anti-P-selectin antibody or a binding fragment thereof, especially crizanlizumab, preferably according to the dosing schedule. In one embodiment the PCR is measured at 12 m after the treatment.

The present invention further provides an anti-P-selectin antibody or a binding fragment thereof, especially crizanlizumab, for use in the treatment or prevention of CKD due to sickel cell nephropathy (SCN), where said patient has eGFR decline not more than 20%, preferably not more than 15%, preferably not more than 10%, preferably not more than 5% compared to the value of eGFR at baseline. In one embodiment the eGFR is measured at 3 m, 6 m, 9 m or 12 m after the treatment of anti-P-selectin antibody or a binding fragment thereof, especially crizanlizumab, preferably according to the dosing schedule. In one embodiment the eGFR is measured at 12 m after the treatment.

The present invention further provides an anti-P-selectin antibody or a binding fragment thereof, especially crizanlizumab, for use in the prevention or slowing the progression of kidney failure due to sickel cell nephropathy (SCN). In one embodiment the patient has eGFR decline not more than 20%, preferably not more than 15%, preferably not more than 10%, preferably not more than 5% compared to the value of eGFR at baseline. In one embodiment the eGFR is measured at 3 m, 6 m, 9 m or 12 m after the treatment of anti-P-selectin antibody or a binding fragment thereof, especially crizanlizumab, preferably according to the dosing schedule. In one embodiment the eGFR is measured at 12 m after the treatment.

In the following, more general expressions are defined in terms of preferred more specific definitions that can replace, individually or as one or more of them, the corresponding terms in invention embodiments, thus forming more preferred invention embodiments.

In other terms: Features, integers, characteristics or compounds, described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

Treatment and/or Prevention of a P-Selectin Mediated Disorder

In the context of the present invention, the term “P-selectin mediated SCN, especially CKD” refers to SCN, especially CKD in which P-selectin plays a role, especially by forming P-selectin/PSGL-1 complexes, in the progression of kidney disease in SCD patients. Often P-selectin mediated SCN, especially CKD is associated with increased levels of P-selectin/PSGL-1 complexes. The anti-P-selectin antibody or binding fragment thereof, especially crizanlizumab, has the ability to reduce the formation of P-selectin/PSGL-1 complexes. It also has the ability to dissociate pre-formed P-selectin/PSGL-1 complexes. Accordingly, it will be appreciated that the use of anti-P-selectin antibodies or binding fragments thereof, especially crizanlizumab or a fragment thereof, allows the (complete or at least partial) prevention of P-selectin mediated SCN, especially CKD, by inhibiting the formation of new P-selectin/PSGL-1 complexes. It will also be appreciated that the use of anti-P-selectin antibodies or binding fragments thereof allows the treatment of existing P-selectin mediated SCN, especially CKD by dissociating pre-formed P-selectin/PSGL-1 complexes. Suitably, the reduction in the formation of P-selectin/PSGL-1 complexes and the dissociation of such complexes occurs during cell to cell interactions.

Specifically administration of crizanlizumab would have a beneficial effect in SCD patients with CKD by blocking P-selectin mediated multicellular adhesion (including leukocytes), and proteinuria, and also reducing vaso-occlusion and potentially its downstream effects in the renal vasculature, which can be clinically demonstrated by a decrease in proteinuria and slowing the decline in glomerular filtration rate (GFR).

SCN, especially CKD, is typically associated with SCD patients with any genotype (HbSS, HbSC, HbSβ⁰-thalassemia and HbSβ⁰+thalassemia). Albuminuria is more common among patients with severe SCD genotypes (HbSS and HbSβ0-thal).

In one embodiment, an anti-P-selectin antibody or binding fragment thereof, especially crizanlizumab or a fragment thereof, is used as a first line treatment for SCN, especially CKD, especially a P-selectin mediated SCN, especially CKD.

In another embodiment an anti-P-selectin antibody or binding fragment thereof, especially crizanlizumab or a fragment thereof, is used in the treatment for SCN, especially CKD, especially a P-selectin mediated SCN, especially CKD, in a patient who has been treated with another medicament against SCN, especially CKD. In another embodiment an anti-P-selectin antibody or binding fragment thereof, especially crizanlizumab or a fragment thereof, is used in combination with another medicament in the treatment for SCN, especially CKD, especially a P-selectin mediated SCN, especially CKD. The term “another medicament” as used herein refers to one or more medicament selected from the list consisting of hydroxyurea, hydroxycarbamide, angiotensin-converting enzyme (ACE) inhibitors, angiotensin II receptor blockers (ARBs).

Angiotensin II receptor blockers (ARBs), also known as angiotensin II receptor antagonists, AT1 receptor antagonists or sartans, are a group of pharmaceuticals that modulate the renin-angiotensin system. Their main uses are in the treatment of hypertension (high blood pressure), diabetic nephropathy (kidney damage due to diabetes) and congestive heart failure. They selectively block the activation of AT1 receptors, preventing the binding of angiotensin II compared to ACE inhibitors. ARBs include but not limited to Losartan, irbesartan, olmesartan, candesartan, valsartan, fimasartan and azilsartan include the tetrazole group (a ring with four nitrogen and one carbon). Losartan, irbesartan, olmesartan, candesartan, and telmisartan include one or two imidazole groups.

Angiotensin-converting enzyme (ACE) inhibitors inhibit the angiotensin-converting enzyme, an important component of the renin-angiotensin system. ACE inhibitors include but not limited to benazepril, zofenopril, perindopril, trandolapril, captopril, enalapril, lisinopril, and ramipril.

It shall be appreciated that there are various routes in which the subject may be provided with an anti-P-selectin antibody or binding fragment thereof. Such suitable routes may be selected from the group consisting of: intravenous, oral, parenteral, intraperitoneal, intramuscular, intravascular, intranasal, intraperitoneal, rectal, subcutaneous, transdermal and percutaneous. More suitably, the patient may be provided with an anti-P-selectin antibody or binding fragment thereof, especially crizanlizumab or a binding fragment thereof, by intravenous route. In one embodiment, the intravenous route is by injection. In another embodiment the patient is provided with anti-P-selectin antibody or binding fragment thereof, especially crizanlizumab or a binding fragment thereof, by subcutaneous route.

The anti-P-selectin antibody or binding fragment thereof, especially crizanlizumab or a binding fragment thereof, may be provided to the subject over any reasonable delivery time. Suitable delivery times may be selected from anywhere between 1 minute to 2 hours, 5 minutes to 90 minutes, 15 minutes to 70 minutes, 20 minutes to 1 hour, or 30 minutes to 50 minutes, for example. In one embodiment, the subject may be provided with an anti-P-selectin antibody or binding fragment thereof over a delivery time of 30 minutes.

Delivery times are suitably applicable to providing the anti-P-selectin antibody or binding fragment thereof by injection, preferably injection intravenously.

In one embodiment, the anti-P-selectin antibody or binding fragment thereof may be provided to the subject by intravenous injection over 30 minutes.

### Loading Phases and Maintenance Phases

Certain aspects of the present invention refer to the provision of an anti-P-selectin antibody or binding fragment, preferably crizanlizumab or a binding fragment thereof, to a subject, especially suffering from or expected to suffer from SCN, especially CKD, and in particular being in need of such treatment, in a loading phase, followed by further provision of the antibody or binding fragment, preferably crizanlizumab or a binding fragment thereof, in a maintenance phase. In such an embodiment, the subject receives a first amount of the antibody or binding fragment over a given period of time during the loading phase, and then receives a lower amount of the antibody or binding fragment over a given period of time, suitably the same given period of time, during the maintenance phase. The different amounts of the antibody required by the loading phase and the maintenance phase may be provided by providing different doses of the antibody and/or by employing different intervals of time between administrations of the antibody. For example, during the maintenance phase the antibody may be provided at essentially the same dose as used during the loading phase, but with longer intervals of time between each incidence of administration. Alternatively, the time between intervals of administration may be the same in each of the loading and maintenance phases, but the dose of antibody provided in each incidence of administration during the maintenance phase may be lower.

Merely by way of example, a suitable loading phase may involve the provision to the subject of a suitable antibody, or binding fragment thereof, in an amount of approximately more than 5 mg/kg, e.g. 10 mg/kg per week of the loading phase (whether this is provided weekly, bi-weekly, or otherwise). In such an embodiment of the invention a suitable maintenance phase may involve the provision to the subject of the antibody, or binding fragment thereof, in an amount of approximately 5 mg/kg per week of the maintenance phase (for example by provision bi-weekly or especially every four weeks). Generally the amount of the antibody, or binding fragment thereof, provided per week of a loading phase may be approximately double that provided per week of the maintenance phase.

As another example, a suitable loading phase may involve the provision to the subject of a suitable antibody, or binding fragment thereof, in an amount of approximately 7.5 or preferably 5 mg/kg bi-weekly, or otherwise. In such an embodiment of the invention a suitable maintenance phase may involve the provision to the subject of the antibody, or binding fragment thereof, in an amount of approximately 7.5 or preferably 5 mg/kg per week of the maintenance phase (for example by provision bi-weekly or especially every four weeks). Generally the amount of the antibody, or binding fragment thereof, provided per week of a loading phase may be approximately double that provided on a per week basis of the maintenance phase.

It will be appreciated that, since the length of the maintenance phase may be much longer than the loading phase, the total amount of the antibody or binding fragment received by the subject over the maintenance phase may be much more than that provided during the relatively shorter loading phase. However, the amount of the antibody that the subject will receive over a set period of the maintenance phase will be lower than the amount that would be received over the same period of the loading phase.

The loading and maintenance phases required by such embodiments of the invention may be put into practice by use of the loading and maintenance doses, and associated administration regimens, considered below.

Various aspects of the invention refer to the average time intervals between maintenance doses, and to average time intervals following the one or more loading doses. It will be recognised that suitable average time intervals may be achieved by varying the number of doses, and the individual intervals between maintenance doses, or following loading doses (whether the loading dose in question is followed by a further loading dose, or a maintenance dose). The following paragraphs provide examples of suitable individual time intervals that may be used in achieving a desired average time interval.

Where +/−3 days is mentioned this may be replaced with +/−2 days, +/−1 day or +/−0 days.

### Determining Effectiveness

In another aspect, the inventors have surprisingly found that providing anti-P-selectin antibodies or binding fragments, preferably crizanlizumab or a binding fragment thereof, to patients with P-selectin mediated disorders may lower the levels of soluble P-selectin in a sample from the patient. The inventors believe that this finding may be of utility in determining and/or monitoring the effectiveness of an anti-P-selectin antibody or binding fragment thereof treatment in a subject with a P-selectin mediated SCN, especially CKD.

Accordingly, the invention also relates to a method of determining effectiveness of treatment with an anti-P-selectin antibody or binding fragment thereof, preferably crizanlizumab or a binding fragment thereof, the method comprising the steps of:
measuring levels of soluble P-selectin in a sample from a subject provided with an anti-P-selectin antibody or binding fragment thereof, preferably crizanlizumab or a binding fragment thereof, and
comparing the subject's soluble P-selectin levels to a reference value, and
thereby determining the effectiveness of the treatment.

In one embodiment, the method is for determining effectiveness of treatment of SCN, especially CKD, with crizanlizumab or a binding fragment thereof in a patient.

Throughout the description and claims of this specification, the singular encompasses the plural unless the context otherwise requires. In particular, where the indefinite article is used, the specification is to be understood as contemplating plurality as well as singularity, unless the context requires otherwise.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

In one aspect the present invention provides an anti-P-selectin antibody or binding fragment thereof, crizanlizumab or a binding fragment thereof, for use in the treatment or prevention of (especially P-selectin mediated) SCN, especially CKD, in a patient, wherein the first two doses of said antibody or binding fragment thereof is provided 2 weeks (+/−3 days) apart followed by further doses provided every 4 weeks (+/−3 days), wherein each dose is between 2.5 mg per kg body weight (2.5 mg/kg) to 20 mg/kg, preferably 2.5 mg/kg to 10 mg/kg, preferably 2.5 mg/kg to 7.5 mg/kg and preferably wherein the interval between the last loading dose and the first maintenance dose is 4 weeks (+/−3 days). In one preferred embodiment, the loading dose is 7.5 mg 7 kg or in particular 5 mg/kg, the maintenance dose is 7.5 mg/kg or in particular 5 mg/kg, and the time interval between the last loading and first maintenance dose is 4 weeks (+/−3 days).

In one embodiment, each of said doses is 2.5 mg/kg. In another particular embodiment, each of said doses is 5 mg/kg. In another embodiment, each of said doses is 7.5 mg/kg. When the dose is initially 7.5 mg/kg, the dose is allowed to be reduced for safety reasons to 5 mg/kg at any time after the loading dose, normally 1 month, 2 months, 3 months, 4 months, 5 months, 6 months, 7 months, 8 months, 9 months, 10 months, or 11 months after the last loading dose. Safety parameters are monitored by health care professionals during the treatment.

The term “anti-P-selectin antibody or binding fragment thereof” as used herein refers to an antibody, or binding fragment thereof, which comprises a P-selectin binding domain. The binding of the antibody (or binding fragment thereof) to P-selectin inhibits the binding of P-selectin to PSGL-1 and thereby reduces the formation of P-selectin/PSGL-1 complexes. Suitably, the anti-P-selectin antibody or binding fragment thereof may reduce the formation of P-selectin/PSGL-1 complexes by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-P-selectin antibody or binding fragment thereof).

Additionally or alternatively, an anti-P-selectin antibody or binding thereof may dissociate preformed P-selectin/PSGL-1 complexes. In a suitable embodiment antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed P-selectin/PSGL-1 complexes. As before, this property may be compared to a suitable control (for example a sample without the presence of an anti-P-selectin antibody or binding fragment thereof).

Additionally or alternatively, an anti-P-selectin antibody or binding thereof may refer to an antibody or binding thereof that is capable of binding to P-selectin specifically, i.e. it binds to P-selectin with an affinity higher than an antibody that is well known not to bind P-selectin specifically. The affinity can be suitably determined by, for example, surface plasmon resonance (BIAcore™) assay. Ideally, the K_{d} of a P-selectin antibody or a fragment thereof is ≤1000 nM, or ≤500 nM, or ≤100 nM, or ≤50 nM, or more preferably by a K_{d}≤25 nM, and still more preferably by a K_{d}≤10 nM, and even more preferably by a K_{d}≤5 nM, or ≤1 nM, or ≤0.1 nM.

In one embodiment, the anti-P-selectin antibody or a binding fragment thereof is crizanlizumab or a binding fragment thereof.

In one embodiment, the anti-P-selectin antibody or (P-selectin) binding fragment thereof may bind P-selectin at any suitable epitope. Suitably, the anti-P-selectin antibody or binding fragment thereof may bind an epitope which is found in the P-selectin lectin-like domain.

In one embodiment, the anti-P-selectin antibody of binding fragment thereof binds P-selectin at amino acid positions 1 to 35 of SEQ ID NO: 1. Suitably the anti-P-selectin antibody or binding fragment thereof binds P-selectin at amino acid positions 4 to 23 of SEQ ID NO: 1. More suitably, the anti-P-selectin antibody or binding fragment thereof binds P-selectin at amino acid positions 4, 14, 17, 21, and 22 of SEQ ID NO: 1.

In one embodiment, the anti-P-selectin antibody or binding fragment thereof comprises a light chain variable region having a CDR sequence selected from the group consisting of KASQSVDYDGHSYMN (SEQ ID NO: 2), AASNLES (SEQ ID NO: 3) and QQSDENPLT (SEQ ID NO: 4).

Specific features of the humanised antibody SelG1, which is a suitable antibody to be employed in the methods and medical uses of the present invention, are set out below.

In a suitable embodiment, the anti-P-selectin antibody or binding fragment thereof may comprise a light chain variable CDR with an amino acid sequence that varies from a sequence selected from the group consisting of KASQSVDYDGHSYMN (SEQ ID NO: 2), AASNLES (SEQ ID NO: 3) and QQSDENPLT (SEQ ID NO: 4) by no more than four amino acid residues, by no more than three amino acid residues, by no more than two amino acid residues, or by no more than one amino acid residue.

In one embodiment the anti-P-selectin antibody or binding fragment thereof comprises a light chain variable region comprising SEQ ID NO: 5. Suitably, the anti-P-selectin antibody or binding fragment thereof comprises of a light chain variable region consisting of SEQ ID NO: 5.

In a suitable embodiment the anti-P-selectin antibody or binding fragment thereof comprises a light chain variable region which comprises or consists of a polypeptide which is at least 90%, at least 95%, at least 96%, at least 97%, at least 98%, or at least 99% identical to SEQ ID NO: 5.

In one embodiment, the anti-P-selectin antibody or binding fragment thereof comprises a heavy chain variable region having a CDR sequence selected from the group consisting of SYDIN (SEQ ID NO: 6), WIYPGDGSIKYNEKFKG (SEQ ID NO: 7) and RGEYGNYEGAMDY (SEQ ID NO: 8).

In a suitable embodiment, the anti-P-selectin antibody or binding fragment thereof may comprise a heavy chain variable CDR with an amino acid sequence that varies from a sequence selected from the group consisting of SYDIN (SEQ ID NO: 6), WIYPGDGSIKYNEKFKG (SEQ ID NO: 7) and RGEYGNYEGAMDY (SEQ ID NO: 8) by no more than four amino acid residues, by no more than three amino acid residues, by no more than two amino acid residues, or by no more than one amino acid residue.

In one embodiment the anti-P-selectin antibody or binding fragment thereof comprises a heavy chain variable region comprising SEQ ID NO: 9. Suitably, the anti-P-selectin antibody or binding fragment thereof comprises a heavy chain variable region consisting of SEQ ID NO: 9.

In a suitable embodiment the anti-P-selectin antibody or binding fragment thereof comprises a heavy chain variable region which comprises or consists of a polypeptide which is at least 90%, at least 95%, at least 96%, at least 97%, at least 98%, or at least 99% identical to SEQ ID NO: 9.

In one embodiment the anti-P-selectin antibody or binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.

In one embodiment the anti-P-selectin antibody or binding fragment thereof comprises a light chain variable region comprising, consisting essentially of or consisting of the sequence SEQ ID NO: 5 and a heavy chain variable region comprising, consisting essentially of or consisting of the sequence SEQ ID NO: 9.

In a suitable embodiment, the antibody or binding fragment thereof may further comprise a constant region. The constant region may comprise a light chain constant region and/or a heavy chain constant region.

The light chain constant region may comprise a human kappa chain or a human lambda chain. Alternatively, the light chain constant region may consist of a human kappa chain or consist of a human lambda chain. Suitably the human kappa chain may be according to SEQ ID NO: 10. Alternatively the human kappa chain may be at least 90%, at least 95%, at least 96%, at least 97%, at least 98%, or at least 99% identical to SEQ ID NO: 10.

The heavy chain constant region may be selected from the group consisting of: IgG, IgA, IgD, IgE, and IgM. Immunoglobulin constant regions may be further classified into isotypes. Thus, the heavy chain constant region may be selected from the group consisting of: IgG₂, IgG₁ IgG₃ and IgG₄.

In one embodiment the heavy chain constant region may comprise an IgG. More suitably, the heavy chain constant region may comprise an IgG₂.

Alternatively the heavy chain constant region may consist of an IgG. More suitably, the heavy chain of the constant region may consist of an IgG₂. Suitably the IgG₂ may be according to SEQ ID NO: 11. Alternatively the IgG₂ may be at least 90%, at least 95%, at least 96%, at least 97%, at least 98%, or at least 99% identical to SEQ ID NO: 11. For example, an IgG₂ sequence to be employed in the invention may comprise five or less, four or less, three or less, two or less, or one or less mutations in IgG₂ sequence according to SEQ ID NO: 11. Suitably, the IgG₂ sequence to be employed in the invention may comprise one mutation in the sequence according to SEQ ID NO: 11. In such an embodiment, the IgG₂ to be employed in the invention suitably has a sequence according to SEQ ID NO: 23. An IgG₂ according to SEQ ID NO: 23 may be desirable in order to further reduce complement activation.

In one embodiment the anti-P-selectin antibody comprises a light chain which is at least 90%, at least 95%, at least 96%, at least 97%, at least 98%, at least 99% identical to SEQ ID NO: 12. Suitably, the anti-P-selectin antibody comprises a light chain according to SEQ ID NO: 12.

In one embodiment the anti-P-selectin antibody comprises a heavy chain which is at least 90%, at least 95%, at least 96%, at least 97%, at least 98%, at least 99% identical to SEQ ID NO: 13. Suitably, the anti-P-selectin antibody comprises a heavy chain according to SEQ ID NO: 13.

In a suitable embodiment the anti-P-selectin antibody comprises a light chain which is at least 90%, at least 95%, at least 96%, at least 97%, at least 98%, at least 99% identical to SEQ ID NO: 12, and a heavy chain which is at least 90%, at least 95%, at least 96%, at least 97%, at least 98%, at least 99% identical to SEQ ID NO: 13. Suitably the anti-P-selectin antibody comprises a light chain according to SEQ ID NO: 12, and a heavy chain according to SEQ ID NO: 13.

Other suitable anti-P-selectin antibodies are disclosed in WO2005/100402, WO1993/021956 and WO1994/025067, which are hereby incorporated by reference in their entirety. In one embodiment, the suitable anti-P-selectin antibody or a fragment thereof is inclacumab or a binding fragment thereof.

In the context of the present invention, the term “binding fragment” as used herein refers to a portion of an antibody capable of binding a P-selectin epitope.

In one embodiment, the binding fragment may comprise an antigen binding and/or variable region. Merely by way of example, a suitable binding fragment may be selected from the group consisting of Fab, Fab′, F(ab′)2, Fv and scFv. Suitable binding fragments may be produced by various methods known in the art. A Fab′ fragment, for example, may be produced by papain digestion of an antibody. A F(ab′)2 fragment, for example, may be produced by pepsin digestion of an antibody.

In one embodiment, the anti-P-selectin antibody or binding fragment thereof, preferably crizalizumab or a binding fragment thereof, has very low immunogenicity. More suitably, the anti-P-selectin antibody, preferably Crizanlizumab, has no or low immunogenicity. The term immunogenicity as used herein, refers to the ability of the antibody or binding fragment thereof to trigger the production of neutralising antibodies against it in the subject.

As mentioned elsewhere in this specification, the generation of neutralising antibodies is highly undesirable, as they may neutralise a therapeutic antibody (or binding fragment thereof), rendering it ineffective. The production of neutralising antibodies may result in a decrease in the levels of the therapeutic antibodies in the subject. Thus, it will be appreciated that a consistent level or amount of therapeutic antibodies in the subject (for example in a serum sample from the subject) may be indicative that no such neutralising antibodies have been produced, and thus that the therapeutic antibody has little or no immunogenicity. By the term consistent it is meant that the level of the therapeutic antibody does not fluctuate in a subject by more than 5%, more than 10%, more that 15%, more than 20%, more than 25%, more than 30%, more than 35%, more than 45%, or more than 50% during the maintenance phase.

### Clinical Trial Protocol:

A Phase II, multicenter, randomized, open label two arm study comparing the effect of crizanlizumab+standard of care to standard of care alone on renal function in sickle cell disease patients ≥16 years with chronic kidney disease due to sickle cell nephropathy

### Purpose and Rationale:

The purpose of this study is to explore the effect of P-selectin inhibition with crizanlizumab on renal function in sickle cell disease (SCD) patients with chronic kidney disease (CKD) who are receiving standard of care medications for SCD and/or CKD, have albuminuria and Stage 1-3a CKD, and have evidence of a rapid decline in their estimated glomerular filtration rate (eGFR).

### Primary Objective:

The primary objective of this study is to evaluate the effect of crizanlizumab standard of care compared to standard of care alone on albuminuria (ACR) decrease at 12 months, as assessed by the proportion of patients with ≥30% decrease in ACR at 12 months compared to baseline. Endpoint: Proportion of patients with *=% or more decrease in ACR at 12 months compared to baseline.

### Secondary Objectives:

To evaluate the effect of crizanlizumab+standard of care compared to standard of care alone on change in albuminuria (ACR), as assessed by the mean change in ACR from baseline to 3, 6, 9, and 12 months of treatment
To evaluate the effect of crizanlizumab+standard of care compared to standard of care alone on albuminuria (ACR) decrease at 6 months, as assessed by the proportion of patients with 30% decrease in ACR at 6 months compared to baseline
To evaluate the effect of crizanlizumab+standard of care compared to standard of care alone on protein to creatinine ratio (PCR) at 12 months, as assessed by the proportion of patients with ≥20% improvement of PCR at 12 months compared to baseline, and the proportion of patients with a stable (within ±20% change) PCR at 12 months compared to baseline
To evaluate the effect of crizanlizumab+standard of care compared to standard of care alone on the percentage change in eGFR, as assessed by the percentage change in eGFR from baseline to 3, 6, 9, and 12 months of treatment
To evaluate the effect of crizanlizumab+standard of care compared to standard of care alone on ACR decline rate, as assessed by the slope of ACR decline from baseline to 12 months of treatment based on ACR values at baseline and at 3, 6, 9, and 12 months
To evaluate the effect of crizanlizumab+standard of care compared to standard of care alone on eGFR decline rate, as assessed by the slope of eGFR decline from baseline to 12 months of treatment based on eGFR values at baseline and at 3, 6, 9, and 12 months
To evaluate the effect of crizanlizumab+standard of care compared to standard of care alone on the progression of CKD at 12 months, as assessed by the proportion of patients with progression of CKD from baseline to 12 months
To evaluate overall safety and, tolerability of crizanlizumab+standard of care compared to standard of care alone, as assessed by the frequency and severity of adverse events (AEs), deaths, serious AEs (SAEs), and laboratory abnormalities

### Population:

This study is designed to enroll approximately 170 male and female patients aged >16 years with CKD due to SCD. Homozygous hemoglobin (Hb) S (sickle cell anemia) (HbSS) and Hb S (HbS) with β-thalassemia (HbSβ0-thal) SCD genotypes are eligible. Eligible patients have an eGFR of ≥45 to ≤120 mL/min/1.73 m2, based on Chronic Kidney Disease Epidemiology Collaboration formula, and an ACR of ≥100 to <2000 mg/g; based on previous studies, this population of patients likely comprise patients with a rapid eGFR decline. Additionally or alternatively, eligible female patients have an eGFR of ≥45 to ≤130 mL/min/1.73 m2 and male patients have an eGFR of ≥45 to ≤140 mL/min/1.73 m2.

### Further Inclusion Criteria

Receiving standard of care drug(s) for SCD and/or CKD. If receiving hydroxyurea (HU)/hydroxycarbamide (HC), angiotensin-converting enzyme (ACE) inhibitor, and/or angiotensin-receptor blocker (ARB) (and still with abnormal ACR despite treatment), must have been receiving the drug(s) for at least 6 months prior to study entry and plan to continue taking the drug(s) at the same dose and schedule until the patient has reached the end of the study. Additionally or alternatively, if receiving HU/HC, ACE inhibitor, and/or ARB (and still with abnormal ACR despite treatment), the patient must have been receiving the drug(s) for at least 3 months prior to study entry and plan to continue taking the drug(s) at the same dose and schedule until the patient has reached the end of the study.
Hb≥4.0 g/dL, absolute neutrophil count ≥1.0×109/L, and platelet count ≥75×109/L
Patients who are clinically stable and are in a non-crisis state

### Exclusion Criteria:

History of stem cell transplant
Patients with evidence of AKI within 3 months of study entry
Blood pressure >140/90 mmHg despite treatment
Patients undergoing hemodialysis
Received blood products within 30 days of Week 1 Day 1
Participating in a chronic transfusion program (pre-planned series of transfusions for prophylactic purposes). Transfusions for acute complications are permitted (acute chest syndrome, acute splenic sequestration, acute hepatic sequestration, worsened anemia)
History of kidney transplant
Patients with hypoalbuminemia

### Study Treatment:

Patients are randomly assigned to one of the following treatment arms in a ratio of 1:1:
Crizanlizumab+standard of care
Standard of care alone

Any of the following drugs that the patient is receiving at study entry is considered the patients standard of care: HU/HC, ACE inhibitors, and ARBs.

The patient continue to take their usual standard of care drugs during the study; thus, there may be some variation in the standard of care regimens used by patients in the study.

Overall, 170 patients are randomized 1:1 to receive either crizanlizumab (as open label medication; 5 mg/kg)+standard of care or standard of care alone. Patients are stratified at randomization based on CKD stage (Stage 1 or Stages 2/3a) and HU/HC prescription (Yes/No). Throughout this document, “study treatment” refers to both treatment arms. Patients randomized to crizanlizumab+standard of care receive crizanlizumab by intravenous (i.v.) infusion over 30 minutes on Week 1 Day 1, followed by a second dose 14 days later (Week 3 Day 1), and then on Day 1 of every 4 weeks for a total on-study treatment period of 12 months in addition to their usual standard of care treatment.

### Efficacy Assessments:

The primary endpoint of this study is the proportion of patients with 30% decrease in ACR at 12 months compared to baseline.
Urine ACR
Urine PCR (Proteine to creainine ratio)
eGFR
Progression of CKD

Three urine samples are collected for each visit time point for which albumin and creatinine concentrations are assessed for ACR.

Albumin is measured by the central laboratory using an immunological assay capable of specifically and precisely quantifying albumin at low concentrations and of producing quantitative results over the clinically relevant range. Albumin concentrations are reported as a ratio to urinary creatinine concentration (mg/g) for each of the three samples. Protein and creatinine concentrations are assessed by the central laboratory. Protein concentrations are reported as a ratio to urinary creatinine concentration (mg/g) for each of the three samples.

The estimated glomerular filtration rate (eGFR) is calculated in the serum from the blood samples collected for assessment of clinical chemistry parameters. eGFR is assessed by the central laboratory. The central laboratory calculates eGFR using the 2009 CKD-EPI formula. The CKD-EPI formula, without the correction for race, used in this study to estimate GFR is as follows:

141×min(Scr/k,1)α×max(Scr/k,1)−1.209×0.993Age

[×1.018 if female], where:
Scr is serum creatinine (in mg/dL)
k is 0.7 for females and 0.9 for males
α is −0.329 for females and −0.411 for males
min is the minimum of Scr/k or 1
max is the maximum of Scr/k or 1

The progression of CKD is assessed based on the blood samples collected for the assessment of eGFR. The progression of CKD is assessed according to the classification presented in KDIGO 2013 based on one or more of the following:
Decline in eGFR category:
   Grade 1 (normal or high), eGFR 90 mL/min/1.73 m2
   Grade 2 (mildly decreased), eGFR 60-89 mL/min/1.73 m2
   Grade 3a (mildly to moderately decreased), eGFR 45-59 mL/min/1.73 m2
   Grade 3b (moderately to severely decreased), eGFR 30-44 mL/min/1.73 m2
   Grade 4 (severely decreased), eGFR 15-29 mL/min/1.73 m2
   Grade 5 (kidney failure), eGFR<15 mL/min/1.73 m2
Rapid progression is defined as a sustained decline in eGFR of more than 5 mL/min/1.73 m2/year

Sequences
P-selectin amino acid sequence
SEQ ID NO: 1
WTYHYSTKAYSWNISRKYCQNRYTDLVAIQNKNEIDYLNKVLPYYSSYYWIGIRKNNKTWTWVGTKKALT
NEAENWADNEPNNKRNNEDCVEIYIKSPSAPGKWNDEHCLKKKHALCYTASCQDMSCSKQGECLETIGNY
TCSCYPGFYGPECEYVRECGELELPQHVLMNCSHPLGNFSFNSQCSFHCTDGYQVNGPSKLECLASGIWT
NKPPQCLAAQCPPLKIPERGNMTCLHSAKAFQHQSSCSFSCEEGFALVGPEVVQCTASGVWTAPAPVCK
CDR light chain amino acid sequence
SEQ ID NO: 2
KASQSVDYDGHSYMN
CDR light chain amino acid sequence
SEQ ID NO: 3
AASNLES
CDR light chain amino acid sequence
SEQ ID NO: 4
QQSDENPLT
Mature light chain variable region amino acid sequence
SEQ ID NO: 5
DIQMTQSPSSLSASVGDRVTITCKASQSVDYDGHSYMNWYQQKPGKAPKLLIYAASNLESGVPSRFSGSG
SGTDFTLTISSLQPEDFATYYCQQSDENPLTFGGGTKVEIKR
CDR heavy chain amino acid sequence
SEQ ID NO: 6
SYDIN
CDR heavy chain amino acid sequence
SEQ ID NO: 7
WIYPGDGSIKYNEKFKG
CDR heavy chain amino acid sequence
SEQ ID NO: 8
RGEYGNYEGAMDY
Mature heavy chain variable region amino acid sequence
SEQ ID NO: 9
QVQLVQSGAEVKKPGASVKVSCKVSGYTFTSYDINWVRQAPGKGLEWMGWIYPGDGSIKYNEKFKGRVTM
TVDKSTDTAYMELSSLRSEDTAVYYCARRGEYGNYEGAMDYWGQGTLVTVSS
Human Kappa constant region amino acid sequence
SEQ ID NO: 10
TVAAPSVFIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
IgG₂ constant region amino acid sequence
SEQ ID NO: 11
ASTKGPSVFPLAPCSRSTSESTAALGCLVKDYFPEPVTVSWNSGALTSGVHTFPAVLQSSGLYSLSSVVT
VTSSNFGTQTYTCNVDHKPSNTKVDKTVERKCCVECPPCPAPPVAGPSVFLFPPKPKDTLMISRTPEVTC
VVVDVSHEDPEVQFNWYVDGMEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
Light chain mature amino acid sequence
SEQ ID NO: 12
DIQMTQSPSSLSASVGDRVTITCKASQSVDYDGHSYMNWYQQKPGKAPKLLIYAASNLESGVPSRFSGSG
SGTDFTLTISSLQPEDFATYYCQQSDENPLTFGGGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSPVT
KSFNRGEC
Heavy chain mature amino acid sequence
SEQ ID NO: 13
QVQLVQSGAEVKKPGASVKVSCKVSGYTFTSYDINWVRQAPGKGLEWMGWIYPGDGSIKYNEKFKGRVTM
TVDKSTDTAYMELSSLRSEDTAVYYCARRGEYGNYEGAMDYWGQGTLVTVSSASTKGPSVFPLAPCSRST
SESTAALGCLVKDYFPEPVTVSWNSGALTSGVHTFPAVLQSSGLYSLSSVVTVTSSNFGTQTYTCNVDHK
PSNTKVDKTVERKCCVECPPCPAPPVAGPSVFLFPPKPKDTLMISRTPEVTCVVVDVSHEDPEVQFNWYV
DGMEVHNAKTKPREEQFNSTFRVVSVLTVVHQDWLNGKEYKCAVSNKGLPAPIEKTISKTKGQPREPQVY
TLPPSREEMTKNQVSLTCLVKGFYPSDIAVEWESNGQPENNYKTTPPMLDSDGSFFLYSKLTVDKSRWQQ
GNVFSCSVMHEALHNHYTQKSLSLSPGK
Light chain complete amino acid sequence
SEQ ID NO: 14
MESQTQVFVYMLLWLSGVDGDIQMTQSPSSLSASVGDRVTITCKASQSVDYDGHSYMNWYQQKPGKAPKL
LIYAASNLESGVPSRFSGSGSGTDFTLTISSLQPEDFATYYCQQSDENPLTFGGGTKVEIKRTVAAPSVF
IFPPSDEQLKSGTASVVCLLNNFYPREAKVQWKVDNALQSGNSQESVTEQDSKDSTYSLSSTLTLSKADY
EKHKVYACEVTHQGLSSPVTKSFNRGEC
Heavy chain complete amino acid sequence
SEQ ID NO: 15
MKCSWVIFFLMAVVTGVNSQVQLVQSGAEVKKPGASVKVSCKVSGYTFTSYDINWVRQAPGKGLEWMGWI
YPGDGSIKYNEKFKGRVTMTVDKSTDTAYMELSSLRSEDTAVYYCARRGEYGNYEGAMDYWGQGTLVTVS
SASTKGPSVFPLAPCSRSTSESTAALGCLVKDYFPEPVTVSWNSGALTSGVHTFPAVLQSSGLYSLSSVV
TVTSSNFGTQTYTCNVDHKPSNTKVDKTVERKCCVECPPCPAPPVAGPSVFLFPPKPKDTLMISRTPEVT
CVVVDVSHEDPEVQFNWYVDGMEVHNAKTKPREEQFNSTFRVVSVLTVVHQDWLNGKEYKCAVSNKGLPA
PIEKTISKTKGQPREPQVYTLPPSREEMTKNQVSLTCLVKGEYPSDIAVEWESNGQPENNYKTTPPMLDS
DGSFFLYSKLTVDKSRWQQGNVFSCSVMHEALHNHYTQKSLSLSPGK
Complete light variable region amino acid sequence
SEQ ID NO: 18
MESQTQVFVYMLLWLSGVDGDIQMTQSPSSLSASVGDRVTITCKASQSVDYDGHSYMNWYQQKPGKAPKL
LIYAASNLESGVPSRFSGSGSGTDFTLTISSLQPEDFATYYCQQSDENPLTFGGGTKVEIKR
Complete heavy variable region amino acid sequence
SEQ ID NO: 19
MKCSWVIFFLMAVVTGVNSQVQLVQSGAEVKKPGASVKVSCKVSGYTFTSYDINWVRQAPGKGLEWMGWI
YPGDGSIKYNEKFKGRVTMTVDKSTDTAYMELSSLRSEDTAVYYCARRGEYGNYEGAMDYWGQGTLVTVS
S
IgG₂ constant region amino acid sequence with a one
amino acid residue mutation to reduce complement activation
SEQ ID NO: 23
ASTKGPSVFPLAPCSRSTSESTAALGCLVKDYFPEPVTVSWNSGALTSGVHTFPAVLQSSGLYSLSSVVT
VTSSNFGTQTYTCNVDHKPSNTKVDKTVERKCCVECPPCPAPPVAGPSVFLFPPKPKDTLMISRTPEVTC
VVVDVSHEDPEVQFNWYVDGMEVHNAKTKPREEQFNSTFRVVSVLTVVHQDWLNGKEYKCAVSNKGLPAP
IEKTISKTKGQPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK

1. A method of treating or preventing sickel cell nephropathy (SCN) in a patient in need of such treatment, comprising administering a pharmaceutically effective amount of an anti-P-selectin antibody or a binding fragment thereof to said patient.

2. The method according to claim 1, wherein the patient is suffering from chronic kidney disease (CKD).

3. The method according to claim 1, wherein the anti-P-selectin antibody is crizanlizumab or a binding fragment thereof.

4. The method according to claim 1, wherein the patient receives co-treatment with one or more standard of care medicament prescribed for CKD.

5. The method according to claim 4, wherein the one or more standard of care medicament prescribed for CKD is selected from the list consisting of hydroxyurea, hydroxycarbamide, angiotensin-converting enzyme (ACE) inhibitors, angiotensin II receptor blockers (ARBs).

6. The method according to claim 1 wherein the anti-P-selectin antibody or a binding fragment thereof is administered as monotherapy.

7. The method according to claim 3 wherein crizanlizumab or a binding fragment thereof is administered as monotherapy.

8. The method according to claim 3, wherein crizanlizumab or a binding fragment thereof is administered 5 mg/kg or 7.5 mg/kg per treatment.

9. The method according to claim 3, wherein crizanlizumab or a binding fragment thereof is administered 5 mg/kg or 7.5 mg/kg monthly.

10. The method according to claim 3, wherein crizanlizumab or a binding fragment thereof is administered in a loading dose followed by a maintenance dose.

11. The method according to claim 10, wherein the loading dose is 5 mg/kg or 7.5 mg/kg and is administered twice in a time interval of 2 weeks+/−3 days, and the maintenance dosage is 5 mg/kg or 7.5 mg/kg and is then administered 4 weeks+/−3 days after the second loading dose administration and then at regular intervals of 4 weeks+/−3 days.

12. The method according to claim 1, wherein said patient has albuminuria (ACR) decrease by at least 30% compared to the ACR level at baseline, after 12 months of treatment.

13. The method according to claim 1, wherein said patient has decrease of PCR by at least 20% compared to the value of PCR at baseline, after 12 months of treatment.

14. The method according to claim 1, wherein said patient has eGFR decline not more than 10% compared to the value of eGFR at baseline, after 12 months of treatment.

15. The method according to claim 1, wherein the anti-P-Selectin antibody or a binding fragment thereof comprises a heavy chain variable region comprising three CDRs comprising, consisting essentially of or consisting of SEQ ID NO: 6, SEQ ID NO: 7, and SEQ ID NO: 8, respectively and a light chain variable region comprising three CDRs comprising, consisting essentially of or consisting of SEQ ID NO: 2, SEQ ID NO: 3, and SEQ ID NO: 4, respectively.
